(19) United States
(12) Patent Application Publication
(10) Pub. No.: US 2026/0248961 A1
(43) Pub. Date: Aug. 27, 2026

(54) UTILIZATION OF A HIBERNATION-LIKE STATE IN NEUROLOGICAL DISEASES

(71) Applicant: OKINAWA INSTITUTE OF SCIENCE AND TECHNOLOGY SCHOOL CORPORATION, Okinawa (JP)

(72) Inventors: Kazumasa NAKAZAWA, Okinawa (JP); Thato Mary MOKHOTHU, Okinawa (JP); Yu-ju LIN, Okinawa (JP); Miyu NAMBU, Okinawa (JP)

(73) Assignee: OKINAWA INSTITUTE OF SCIENCE AND TECHNOLOGY SCHOOL CORPORATION, Okinawa (JP)

(21) Appl. No.: 18/879,494

(22) PCT Filed: May 31, 2023
(86) PCT No.: PCT/JP2023/020392

§ 371 Date: Dec. 27, 2024

Related U.S. Application Data
(60) Provisional application No. 63/356,600, filed on Jun. 29, 2022.

(51) Int. Cl. A61K 48/00; A61K 31/5513; A61K 38/16; A61K 38/17; A61P 25/08

(52) U.S. Cl. CPC A61K 48/005; A61K 31/5513; A61K 38/16; A61K 38/177; A61P 25/08

(57) ABSTRACT

The purpose of this invention is to provide a new treatment method for epilepsy that solves the problems of current epilepsy treatment and has long-term effects. The present invention is a method of treating a disease, comprising inducing hypothermia or hypometabolism in a subject. Thermoregulatory or metabolic regulatory neurons are stimulated in the step of inducing hypothermia or hypometabolism in the subject. Q neurons are stimulated in the step of inducing hypothermia or hypometabolism in the subject.

### TECHNICAL FIELD

The present invention relates to the utilization of a hibernation-like state in neurological diseases.

### BACKGROUND ART

Temporal Lobe Epilepsy (TLE) is the most commonly medically refractive focal epilepsy (NPL1: Engel, 2014: Annals of Indian Academy of Neurology, 2014, 17(Suppl 1), S12-S17. https://doi.org/10.4103/0972-2327.128644). It mainly begins in the limbic areas and could be secondarily generalized, resulting in tonic-clonic seizures (NPL2: Shibley and Smith, 2002: Epilepsy Research, 2002, 49(2), 109-120. doi:10.1016/s0920-1211(02)00012-8; NPL3: Bone et al., 2012: Epilepsia. 2012 May; 53(5):817-24. doi: 10.1111/j.1528-1167.2012.03435.x.). The distinctive three phases of TLE are (1) Acute phase: in which there is a prolonged seizure due to a brain insult, (2) Latent phase: is a period between the initial precipitating (IPI) and the chronic phase without any exhibition of behavioral seizures, and (3) The chronic phase: is marked by spontaneous recurrent seizures (SRS) (NPL4: Panayiotopoulos, 2005: The Epilepsies: Seizures, Syndromes and Management. Oxfordshire (UK): Bladon Medical Publishing; 2005. Chapter 4, Principles of Therapy in Epilepsies; NPL5: Curia et al., 2008: Journal of Neuroscience Methods, 172(2), 143-157. doi:10.1016/j.jneumeth.2008.04.019; NPL6: Kandratavicius et al., 2014: Neuropsychiatric Disease and Treatment, 1693. doi:10.2147/ndt.s50371; NPL7: Janz et al., 2017: Elife. 2017 Jul. 26; 6:e25742. doi: 10.7554/eLife.25742.). These are seemingly unprovoked seizures in people with epilepsy, which occur weeks, months, or even years after the first incident (NPL8: Maguire, 2016: Epilepsy Currents, 2016, 16(1), 31-33. https://doi.org/10.5698/1535-7597-16.1.31). In human cases, epilepsy is usually diagnosed in the chronic state, although the consensus is that many neurological changes occur long before the start of the observable recurrent seizures (NPL9: Cho et al., 2015: Nature Communications, 6(1). doi:10.1038/ncomms7606; NPL8: Maguire, 2016: Epilepsy Currents, 16(1), 31-33. https://doi.org/10.5698/1535-7597-16.1.31).

Epileptogenesis, the development of epilepsy from one stage to the other, is still under intense investigation. There is currently an exciting debate in the epilepsy field: One school of thought is that the IPI presents all the necessary features to elicit SRS, and even without a latent phase, chronic seizures will begin (NPL10: Rattka et al., 2011: Neuropharmacology, 60(2-3), 505-512. doi:10.1016/j.neuropharm.2010.11.005; NPL11: Loscher et al., 2015: Epilepsy & Behavior, 52, 78-92. https://doi.org/10.1016/j.yebeh.2015.08.037). This view is based on observing varied results from some human and animal studies in which some seizures began almost immediately after the IPI (NPL11: Loscher et al., 2015: Epilepsy & Behavior, 52, 78-92. https://doi.org/10.1016/j.yebeh.2015.08.037). Another thought is that latency is a prerequisite process in epileptogenesis during which changes occurring in the brain, including circuit rewiring, will culminate in the seizures elicited in chronic periods (NPL4: Panayiotopoulos, 2005: The Epilepsies: Seizures, Syndromes and Management. Oxfordshire (UK): Bladon Medical Publishing; 2005. Chapter 4, Principles of Therapy in Epilepsies; NPL12: Lee et al., 2017: Experimental Neurology, 296, 89-98. doi:10.1016/j.expneurol.2017.07.007). This is based on a traditional view of the stages of epilepsy, whose central gap is the lack of explicit knowledge about brain activity in the absence of seizures. Maguire (2016) (NPL8) suggests that many cellular and molecular changes occur in the brain, making the emergence of SRSs more likely. It is now appreciated that dubbing the latent period as the “silent period” may be misleading: While behaviorally, there are no seizures, the brain activity may show a different picture.

The quest to suppress seizures has led to several brain regions being studied and pinpointed as possibilities for therapeutic targets. It is natural to look to the hippocampus or the amygdala first, as these are usually the seizure generation sites in TLE (NPL13: Bertram, 2009: Epilepsy Behav. 2009 January; 14 Suppl 1(Suppl 1):32-7. doi: 10.1016/j.yebeh.2008.09.017.). Since seizures are characterized by synchronous, high-frequency discharges, some medications seek to block ion channels found on excitable cells, such as voltage-gated sodium channels. Since their fast-opening leads to membrane depolarization, drugs such as valproate or phenytoin, which block these channels, counteract this activity, either by transiently blocking them or by lengthening their inactive state (NPL14: Beck and Yaari, 2012: Jasper's Basic Mechanisms of the Epilepsies [Internet]. 4th ed. Bethesda (MD): National Center for Biotechnology Information (US); 2012).

Many drugs, such as benzodiazepines have been developed have addressed the chemical balance issue between GABA and glutamate by enhancing sensitivity to GABA: They bind to GABAA receptors which in turn increases the rate of chloride or other negative ions coming into the cell, reducing activity (NPL15: Nicholson et al., 2018: Mol Psychiatry. 2018 September; 23(9):1851-1867. doi: 10.1038/s41380-018-0100-y.). Other drugs like barbiturates also have this GABAA induced sedative effect, but additionally, they directly block AMPA and Kainate receptors, as well as reducing release of glutamate (NPL16: Loscher & Rogawski, 2012: Epilepsia. 2012 December; 53 Suppl 8:12-25. doi: 10.1111/epi.12025.). These are the mainstays of epileptic treatment. Some medication is thought to work through the inhibition of the high threshold calcium channels. Once inhibited, presynaptic calcium levels do not rise when action potentials arrive, which subsequently inhibits neurotransmitters' release. This is an indirect method in which glutamate release could be reduced. On the other hand, inhibition of low threshold calcium channels in postsynaptic elements is a course of action to reduce postsynaptic excitability (NPL14: Beck and Yaari, 2012: Jasper's Basic Mechanisms of the Epilepsies [Internet]. 4th ed. Bethesda (MD): National Center for Biotechnology Information (US); 2012).

One can infer from the above that since these medications target receptors and ion channels, they do not only affect the activity of the seizure focus but may cause effects on nearby or even distant regions of the brain. Numerous side effects have been documented in association with the use of anti-seizure medication. Many people experience these side effects, especially at the beginning of treatment. A study performed at Yale University with a sample of over four thousand adult patients of epilepsy, of which 70% had focal epilepsy, reported that 15-20% of them experienced psychiatric and behavioral side effects because of their medication. These included mood swings, bouts of depression, psychosis, irritability, and aggressive tendencies, and depending on the severity and tolerance the patient might have, these can lead to nonadherence to the medication. Amongst the medications that the patients were taking, all of them were associated with side effects. However, two particularly had the highest rates of side effects, Zonisamide and Levetiracetam, and interestingly, in all the medications, the load was not a factor in predicting side effect rate (NPL17: Chen et al., 2017: Epilepsy Behav. 2017 November; 76:24-31. doi: 10.1016/j.yebeh.2017.08.039.).

There are not many alternative therapies to seizure medication. Dietary changes such as adopting the ketogenic diet, or the modified Atkins diet can be prescribed to patients. Their effectiveness is limited, and their studies are mostly small and non-replicated, and they work best as an add-on rather than a replacement of pharmaceutical intervention. Some populations of patients, e.g., young children, also seem to benefit more from dietary interventions for their epilepsy than adults (NPL18: Loscher et al., 2020: Pharmacol Rev. 2020 July; 72(3):606-638. doi: 10.1124/pr.120.019539.).

In some cases of refractory epilepsy, respective surgery is presented as an option. This requires that the seizure be focal and always gets generated in the same region, and this area is then removed. This procedure is not readily accessible globally because of lack of expertise and/or funds. It cannot be used on all patients as it is incredibly invasive, and the eligibility criteria are strict (NPL19: Jobst & Cascino, 2015: JAMA. 2015 Jan. 20; 313(3):285-93. Doi: 10.1001/jama.2014.17426.). For instance, the patient needs to have a high incidence of seizures and show that at least two therapy strategies have failed to control these seizures (NPL19: Jobst & Cascino, 2015: JAMA. 2015 Jan. 20; 313(3):285-93. doi: 10.1001/jama.2014.17426.). In a case study of 118 patients, the seizure-free outcome was 58% compared to 8% in chronic medication, and several meta-analyses featured in the same report seem to have results that align with this trend. Therefore, surgeries are better control measures for seizures than medication, and yet even surgeries can still fail and patients may relapse due to many factors surrounding their condition and the surgical procedure (NPL20: Harroud, 2012: Epilepsy Res Treat. 2012; 2012:201651. doi: 10.1155/2012/201651.). Given all the above, one can conclude that there is a desperate need to develop more alternatives for therapy and continue to build up towards finding a cure for this debilitating disease soon.

There was an attempt to cure seizures by lowering the body temperature with ice or surgically cooling down the brain. In their study in two chimpanzees, researchers found that during a hypothermic state, it was harder for seizures to be elicited through electrostimulation. However, this impact was only effective during the intervention. It was shown in the same study that during rewarming, one of the chimpanzees had an electrographic seizure. Both animals elicited seizures upon repetitive electrical stimulation after the hypothermia treatment (NPL21: Bawldwin et al., Science, 1956 Nov. 9; 124(3228):931-2). In humans, following failed respective surgeries, there have been some trials to help patients achieve some freedom from seizures through local brain cooling. Karlov describes a procedure utilising ethyl chloride on the epileptogenic zone with complete remission from seizures even after decades of monitoring (NPL22: Epilepsia, 2003 December; 44(12):1605). On one hand, the first trial with global body cooling using ice packs and a water mattress only proved effective for treatment in the short term, and on the other hand, the use of ethyl chloride required restricted administration to the seizure focus, which isn't always known or limited to one area in TLE (NPL23: GUNMA UNIVERSITY Press release Oct. 30, 2019, https://www.gunma-u.ac.jp/wp-content/uploads/2019/10/R011030-press.pdf).

One of the biggest problem with current epilepsy treatments is their short-term effectiveness. Even when patients find a drug that effectively suppresses seizures, they easily relapse when they stop taking it. The same problem exists with modern brain activity manipulation, such as the cooling method described above.

Patent literature 1 also discloses an invention of a method for inducing a hypometabolic state (preferably a hibernation state) in a subject with a disease and an apparatus for this purpose (PL1: WO2022/210833). However, in this patent literature, the mechanism of inducing a Q neuron-induced hypothermia/hypometabolism (QIH) state is clarified using mice, but the effect of treating the disease by inducing a Q neuron-induced hypothermia/hypometabolism (QIH) state is neither examined nor confirmed in any way.

### CITATION LIST

### Patent Literature

[PL 1] WO2022/210833

### Non Patent Literature

[NPL 1] Annals of Indian Academy of Neurology, 2014, 17(Suppl 1), S12-S17. https://doi.org/10.4103/0972-2327.128644
[NPL 2] Epilepsy Research, 2002, 49(2), 109-120. doi:10.1016/s0920-1211(02)00012-8;
[NPL 3] Epilepsia. 2012, May; 53(5):817-24. doi: 10.1111/j.1528-1167.2012.03435.x.
[NPL 4] The Epilepsies: Seizures, Syndromes and Management. Oxfordshire (UK): Bladon Medical Publishing; 2005. Chapter 4, Principles of Therapy in Epilepsies;
[NPL 5] Journal of Neuroscience Methods, 172(2), 143-157. doi:10.1016/j.jneumeth.2008.04.019
[NPL 6] Neuropsychiatric Disease and Treatment, 2014, 1693. doi:10.2147/ndt.s50371;
[NPL 7] Elife. 2017 Jul. 26; 6:e25742. doi: 10.7554/eLife.25742.
[NPL 8] Epilepsy Currents, 2016, 16(1), 31-33. https://doi.org/10.5698/1535-7597-16.1.31
[NPL 9] Nature Communications, 6(1). doi:10.1038/ncomms7606
[NPL 10] Neuropharmacology, 60(2-3), 505-512. doi:10.1016/j.neuropharm.2010.11.005;
[NPL 11] Epilepsy & Behavior, 52, 78-92. https://doi.org/10.1016/j.yebeh.2015.08.037
[NPL 12] Experimental Neurology, 296, 89-98. doi:10.1016/j.expneurol.2017.07.007
[NPL 13] Epilepsy Behav. 2009 January; 14 Suppl 1:32-7. doi: 10.1016/j.yebeh.2008.09.017.
[NPL 14] Jasper's Basic Mechanisms of the Epilepsies [Internet]. 4th ed. Bethesda (MD): National Center for Biotechnology Information (US); 2012.
[NPL 15] Mol Psychiatry. 2018 September; 23(9):1851-1867. doi: 10.1038/s41380-018-0100-y.
[NPL 16] Epilepsia. 2012 December; 53 Suppl 8:12-25. doi: 10.1111/epi.12025.
[NPL 17] Epilepsy Behav. 2017 November; 76:24-31. doi: 10.1016/j.yebeh.2017.08.039.
[NPL 18] Pharmacol Rev. 2020 July; 72(3):606-638. doi: 10.1124/pr.120.019539.
[NPL 19] JAMA. 2015 Jan. 20; 313(3):285-93. Doi: 10.1001/jama.2014.17426.
[NPL 20] Epilepsy Res Treat. 2012; 2012:201651. doi: 10.1155/2012/201651.
[NPL 21] Science, 1956 Nov. 9; 124(3228):931-2
[NPL 22] Epilepsia, 2003 December; 44(12):1605
[NPL 23] GUNMA UNIVERSITY Press release Oct. 30, 2019, https://www.gunma-u.ac.jp/wp-content/uploads/2019/10/R011030-press.pdf[Summary of Invention]

### SUMMARY OF INVENTION

### Technical Problem

One of the biggest problem with current epilepsy treatments is their short-term effectiveness. Even when patients find a drug that effectively suppresses seizures, they easily relapse when they stop taking it. The same problem exists with modern brain activity manipulation, such as the cooling method described above.

The purpose of this invention is to provide a new treatment method for epilepsy that solves the problems of current epilepsy treatment and has long-term effects.

### Solution to Problem

To address these challenges, the current invention utilizes a hibernation-like hypometabolic state by manipulating a specific subpopulation of neurons in the hypothalamus. As one such approach, our study used recently developed transgenic mice to induce a hibernation-like hypometabolic state (Q-neuron-induced hypothermia and hypometabolism, QIH) to lower the temperature and reduce the metabolic rate of the brain (Nature. 2020 July; 583(7814):109-114. doi: 10.1038/s41586-020-2163-6., Cell Rep Methods. 2022 Nov. 14; 2(11):100336. doi: 10.1016/j.crmeth.2022.100336.). While this hibernation-like state induces substantial decreases in body temperature, metabolism, behavioral activity, and food intake, the recovery from QIH does not accompany any damage in the tissues, including the brain, heart, kidney, liver, and muscle, suggesting a possibility of practical use of QIH for therapeutic purposes. Conservation of the Q-neurons in the hypothalamus across many mammalian species potentially generalizes the application of the current intervention. The current intervention particularly aims to further characterize the impact of QIH on the neuronal physiology and anatomical morphology in the brain, determine if it compromises memory retention, and reveal its effect on the development of TLE. To address these issues, the current invention may utilize torpor-like state as hypothermic and/or hypometabolic state. The method of inducing the torpor-like state is not limited, but can be induced, for example, by applying ultrasound stimulation (Nature. metabolism vo. 5, 789-803, 2023). In short, the gist of the invention is as follows.

The present invention includes the following embodiments:
(1) A method of treating a disease, comprising inducing hypothermia or hypometabolism in a subject.
(2) The method of treating a disease according to (1), wherein thermoregulatory or metabolic regulatory neurons are stimulated in the step of inducing hypothermia or hypometabolism in the subject.
(3) The method of treating a disease according to (2), wherein a Q neuron is stimulated in the step of inducing hypothermia or hypometabolism in the subject.
(4) The method of treating a disease according to any one of (1) to (3), wherein the disease is a neurological disease.
(5) The method of treating a disease according to (4), wherein the neurological disease is epilepsy.
(6) The method of treating a disease according to (5), wherein the epilepsy is temporal lobe epilepsy (TLE).
(7) The method of treating a disease according to (5) or (6), wherein the step of inducing hypothermia or hypometabolism in the subject is performed during a latent phase of the disease.
(8) The method of treating a disease according to any one of (5) to (7), wherein the treating comprises delaying a seizure.
(9) The method of treating a disease according to any one of (5) to (8), wherein the treating comprises alleviating a seizure.
(10) The method of treating a disease according to any one of (1) to (9), wherein the subject is a mammal.
(11) A kit for treating a neurological disease, comprising a recombinant vector comprising a gene encoding a protein that functions to specifically stimulate thermoregulatory or metabolic regulatory neurons and/or a compound for activating the thermoregulatory or metabolic regulatory neurons.
(12) The kit for treating a neurological disease according to (11), wherein the thermoregulatory or metabolic regulatory neurons are Q neurons.
(13) The kit for treating a neurological disease according to (12), comprising the recombinant vector with a hM3Dq gene and a Qrfp (pyroglutamylated RFamide peptide) promoter, and the compound of clozapine-N-oxide.
(14) The kit for treating a neurological disease according to (12), comprising the recombinant vector with a channelrhodopsin gene and a Qrfp (pyroglutamylated RFamide peptide) promoter for specifically stimulating Q neurons.
(15) A kit for treating a neurological disease, comprising a compound for stimulating thermoregulatory or metabolic regulatory neurons in a subject and a recombinant vector comprising a gene encoding a receptor for specifically stimulating the thermoregulatory or metabolic regulatory neurons in response to the compound.
(16) A kit for treating a neurological disease, comprising a recombinant vector comprising a gene encoding a protein, preferably a membrane protein, more preferably a channelrhodopsin, for specifically stimulating thermoregulatory or metabolic regulatory neurons in a subject in response to light, preferably infrared light.

### Advantageous Effects of Invention

According to the present invention, by stimulating the thermoregulatory or metabolic regulatory neurons in a subject to induce hypothermic and/or hypometabolic state the recurrence of epileptic seizures can be effectively suppressed and the period until recurrence can be significantly prolonged. The hypothermic and/or hypometabolic state may include, for example the so-called hibernation state, hibernation-like state, torpor state, torpor-like state. In addition, according to the present invention, epileptic seizures can be effectively alleviated. Inducing hypothermic state or hypometabolic state does not influence sensory perception, memory recall, and its behavioral expression. This method of treatment is effective as a novel treatment method for diseases such as epilepsy.

### BRIEF DESCRIPTION OF DRAWINGS

FIG. 1A Physiological changes in the brain during QIH. A representative time course plot of the change of the body temperature during QIH. X-axis corresponds to the time after CNO injection (QIH induction).

FIG. 1B Physiological changes in the brain during QIH. Changes in the power spectrum density in the dorsal CA1 of the hippocampus. Each plots are power estimates from the LFP traces averaged over 30 minutes of recording after QIH induction.

FIG. 1C Physiological changes in the brain during QIH. Changes in the mean firing rates of single units recorded from the dorsal CA1 of the hippocampus. Left, bar graphs averaging spike rates from pre-QIH induction (baseline), 0-2 hours, and 2-4 hours after QIH induction. Right, the time course plots of firing rates over 1 minute bins.

FIG. 1D Physiological changes in the brain during QIH. Changes in the mean firing rates of multi unit recorded from the hypothalamic regions. X-axis corresponds to the time after QIH induction.

FIG. 2A Electrophysiological properties in the hippocampus during a seizure episode, and the influence of QIH on the development of epilepsy. A representative LFP trace from pre-seizure induction (baseline) and during a seizure episode. Seizure activity is characterized by intermittent population spikes (single seizures) and bursts of such aberrant activity (prolonged seizures).

FIG. 2B Electrophysiological properties in the hippocampus during a seizure episode, and the influence of QIH on the development of epilepsy. Duration of the latent phase in the animals that had 2 days of QIH period after seizure induction (QIH) and those without QIH (No QIH).

FIG. 2C Electrophysiological properties in the hippocampus during a seizure episode, and the influence of QIH on the development of epilepsy. Racine scale showing the severity of SRS in the animals with or without QIH during the latent phase.

FIG. 3A QIH did not affect the previously acquired contextual fear memory. A. The experimental outline of the behavioral procedures (see materials and methods for details).

FIG. 3B QIH did not affect the previously acquired contextual fear memory. Freezing behaviors during the conditioning and testing (pre/post-QIH) sessions in the animals that underwent QIH or the controls without QIH.

### DESCRIPTION OF EMBODIMENTS

The following is a detailed description of the present invention. In this specification, molecular biological techniques can be performed by methods described in general experimental manuals known to those skilled in the art or by methods similar thereto, unless otherwise specified. In addition, terms used herein are to be interpreted in the sense normally used in the art, unless otherwise noted.

Unless otherwise noted, all terms in the present invention have the same meaning as commonly understood by one with ordinary skill in the art to which this disclosure belongs. The singular terms “a,” “an,” and “the” include plural referents unless context indicates otherwise. Similarly, the word “or” is intended to include “and” unless the context indicates otherwise. In this specification, molecular biological techniques can be performed by methods described in general experimental manuals known to those skilled in the art or by methods similar thereto, unless otherwise specified.

### Method of Treating a Disease

According to the present invention, by stimulating the thermoregulatory neurons or metabolic regulatory neurons in a subject, a hypothermic or hypometabolic state is induced, and a therapeutic effect can be obtained for diseases. The above diseases include neurological diseases. According to the present invention, the recurrence of seizures in epilepsy can be effectively suppressed and the period until recurrence can be markedly lengthened. In addition, according to the present invention, epileptic seizures can be effectively alleviated. The present method of treatment is effective as a new treatment method for diseases such as epilepsy. The hypothermic and/or hypometabolic state may include, for example the so-called hibernation state, hibernation-like state, torpor state, torpor-like state. In the present application, “hypothermic state” and “hypothermia”, and “hypometabolic state” and “hypometabolism” are used with the same meaning.

A method of treating a disease of the present invention comprises inducing hypothermia or hypometabolism in a subject.

The subject includes mammals, specifically humans, mice, rats, dogs, cats, hamsters, rabbits, monkeys, gorillas, chimpanzees, orangutans, and the like.

Hypothermia or hypothermic state refers to a state in which the subject's body temperature is below normal, e.g., if the normal body temperature is 37° C., the body temperature ranges from 20° C. to 35° C., 22° C. to 33° C., and 24° C. to 30° C. Hypometabolism or Hypometabolic state refers to a state in which the subject's body functions are reduced, e.g., reduced heart rate, weak respiration, low-potential EEG, and many other physiological parameters show reduced function. In the present invention, hypothermic or hypometabolic state may include a so-called hibernation state, hibernation-like state, torpor state and torpor-like state.

As used herein, “hibernation” is a hypothermic and hypometabolic state observed in mammals. “Torpor” is a short-term hypometabolic state. Hibernation differs from daily torpor in that in daily torpor, there is little or no decrease in TR (theoretical temperature set point (° C.)) and a decrease in H (heating efficiency), whereas in hibernation, both TR and H are significantly decreased. In this specification, “hibernation-like state” means a state in which both TR and H decrease significantly with a decrease in TA (ambient temperature of the subject (° C.)). “Torpor-like state” means a state in which there is little or no decrease in TR and a decrease in H.

The method used to induce hypothermia or a hypometabolic state in the subject is not particularly limited in this method of treatment.

Preferred methods of inducing hypothermia or hypometabolic state in a subject in the treatment method of the present invention include, for example, stimulation of thermoregulatory neurons or metabolic regulatory neurons.

The method of stimulating the above thermoregulatory neurons or metabolic regulatory neurons is not limited as long as the method is capable of stimulating excitatory stimuli or disinhibitory stimuli specifically to these neurons. For example, a method using voltage which is applied to the above neurons using deep brain electrodes, etc., a method using a compound that can induce excitatory stimulation or disinhibitory stimulation specifically in the above neurons, a method using the DREADD method to express a receptor (e.g., hM3Dq) in the above neurons and to administer a ligand for the receptor (e.g., clozapine-N-oxide (CNO)), a method of stimulating by light by expressing a photoreceptor (channelrhodopsin) in the above neurons, etc. are exemplified. In addition, the method of inducing the torpor-like state is not limited, but can be induced, for example, by applying ultrasound stimulation (Nature. metabolism vo. 5, 789-803, 2023).

Q-neurons are preferred examples of the above thermoregulatory or metabolic regulatory neurons. Herein, “Q-neurons” are neurons that are present in an inner region of the hypothalamus, namely, the regions of the anteroventral periventricular nucleus (AVPe), the medial preoptic area (MPA), and the periventricular nucleus (Pe), and these Q-neurons produce pyroglutamylated RFamide peptide (QRFP). The pyroglutamylated RFamide peptide (QRFP) is a neuropeptide identified as an endogenous ligand of the GPR103 receptor. QRFPs are strongly expressed in the hypothalamus and are thought to be involved in the regulation of sleep and wakefulness, as such neurons have been shown to clearly have an effect of enhancing the arousal system.

A hibernation-like state can be induced in a subject that is living, non-hibernating animals by applying, in the brain of the subject, an excitatory stimulus or a disinhibitory stimulus to pyroglutamylated RFamide peptide (QRFP)-producing neurons in the region of the hypothalamus from the anteroventral periventricular nucleus (AVPe), the medial preoptic area (MPA) or the periventricular nucleus (Pe).

In the present invention, pyroglutamylated RFamide peptide (QRFP)-producing neurons can be stimulated using a specific device. In the present invention, a voltage can be loaded onto QRFP-producing neurons, thereby stimulating the QRFP-producing neurons. The excitatory stimuli or the disinhibitory stimuli can be triggered by stimulation using a deep brain electrode or by stimulation using an activator of QRFP-producing neurons.

In the present invention, a stimulus can be applied to the QRFP-producing neurons by expressing, specifically in the QRFP-producing neurons, a receptor (e.g., hM3Dq) using the DREADD method, and administering a ligand (e.g., clozapine-N-oxide (CNO)) for the receptor thereof. The hM3Dq can be expressed in the QRFP-producing neurons by infecting the QRFP-producing neurons of the subject with a virus (e.g., adenovirus, adeno-associated virus, etc.) having a gene encoding hM3Dq, which is operably linked to a QRFP promoter. A specific example is shown in the Example of this application.

An optogenetic approach may also be used in this invention. Specifically, photosensitive proteins (e.g., channelrhodopsin) can be expressed, especially in QRFP-producing neurons, and stimulated by light irradiation. A virus (e.g., adenovirus, adeno-associated virus, etc.) having a gene encoding a channelrhodopsin operably linked to a QRFP promoter can be used to express channelrhodopsin by infecting QRFP-producing neurons of the target.

Specifically, an optogenetic method using modified human opsin4 (OPN4; also known as melanopsin), a G protein-coupled-receptortype blue-light photoreceptor was developed. C-terminally truncated OPN4 (OPN4dC) stably and reproducibly induces QIH (Q-neuron-induced hypothermia and hypometabolism) for at least 24 h by illumination with low-power light (3 mW, 473 nm laser) with high temporal resolution. The high sensitivity of OPN4dC allows us to transcranially stimulate Q neurons with blue-light-emitting diodes and non-invasively induce the QIH (Cell Reports Methods 2, 100336, Nov. 21, 2022).

In the present invention, recombinant vectors that function to specifically stimulate thermoregulatory or metabolic regulatory neurons of the subject can be used. As such recombinant vectors, those having the above hM3Dq gene and Qrfp (pyroglutamylated RFamide peptide) promoter, channelrhodopsin gene and Qrfp (pyroglutamylated RFamide peptide) promoter are preferred examples.

A torpor state or torpor-like state can be induced in a subject by remote transcranial ultrasound stimulation at the hypothalamus preoptic area (POA) (Nature. metabolism vo. 5, 789-803, 2023). In addition, the torpor state or the torpor-like state may be induced in a manner similar to the Q neuron stimulation method described above. Ultrasound stimulation may be used as a method of Q neuron stimulation.

The activator can be screened using QRFP neurons or can be searched for using cultured cells in which receptors that express on QRFP neurons have been forcibly expressed. A neuron activator may be administered locally to QRFP-producing neurons using an applicator. The QRFP-producing neuron-specific activator may be administered through intracerebroventricular administration, intrathecal administration, or systemic administration such as intravenous administration.

The method of the present invention is particularly effective for neurological diseases, and is particularly suitable for epilepsy such as temporal lobe epilepsy (TLE).

In the method of treatment of the present invention, the process of inducing hypothermia or hypometabolic state in the subject above should be performed at an appropriate time, depending on the disease, and may be performed only once or multiple times.

When the method of treatment is used for epilepsy, it is preferred that the process of inducing hypothermia or a hypometabolic state in the subject is performed during a latent phase of the disease. When the process of inducing hypothermia or hypometabolic state in the subject above is performed during the latent phase of the disease, the recurrence of epileptic seizures can be effectively suppressed and the period until recurrence can be significantly extended.

When the subject is a human, the process of inducing hypothermia or hypometabolic state should preferably be performed within six months, four months, three months, two months, one month, or two weeks of the acute attack in the acute phase. Considering the length of the latent period in the average human, it is more preferable to perform the process within one to three months of the acute attack in the acute phase, and even more preferable within two months. When the process of inducing hypothermia or hypometabolic state is performed multiple times when the subject is human, the frequency is once every six months to once every ten years, once every one to eight years is preferred, once every two to six years is more preferred, and once every four to five years is even more preferred. In addition, the process of inducing hypothermia or hypometabolic state is performed at 10° C. to 20° C., preferably at around 15° C.

When the subject is a small animal such as a dog or a cat, the process of inducing hypothermia or hypometabolic state should preferably be performed within 2 months, 1 month, 3 weeks, 2 weeks, 1 week, or 2-3 days after the acute attack in the acute phase. Considering the length of the latent period in the average small animal, it is more preferable to do so within 1-3 weeks of the acute attack in the acute phase, and even more preferable to do so within 2 weeks. The frequency should be once every 2 months to 3 years, preferably once every 6 months to 2 years, and once a year is even more preferable. In addition, the process of inducing hypothermia or hypometabolic state is performed at 10° C. to 20° C., preferably at around 15° C.

### A Kit for Treating a Neurological Disease

The present invention includes a kit for treating a neurological disease, comprising the materials necessary to realize the above-mentioned method of treating diseases of the invention.

An embodiment of the present invention is a kit for treating a neurological disease comprising a recombinant vector that functions to specifically stimulate thermoregulatory neurons or metabolic regulatory neurons in a subject, and/or a compound for stimulating thermoregulatory neurons or metabolic regulatory neurons. It is preferred that the above recombinant vector contains excitatory DREADD (hM3Dq) gene and Qrfp (pyroglutamylated RFamide peptide) promoter, and the above compound is clozapine-N-oxide. It is also preferred that the above recombinant vector is a recombinant vector having channelrhodopsin gene and Qrfp (pyroglutamylated RFamide peptide) promoter.

It can also be said that an embodiment of the present invention is a kit for treating a neurological disease, comprising a compound for stimulating thermoregulatory or metabolic regulatory neurons of a subject and a recombinant vector containing a gene encoding a receptor for specifically stimulating thermoregulatory or metabolic regulatory neurons in response to the compound. With the present kit for treating a neurological disease, for example, a viral recombinant vector (e.g., adenovirus, adeno-associated virus, etc.) with a gene encoding hM3Dq operably linked to the QRFP promoter can be used to specifically express hM3Dq on QRFP-producing neurons. The QRFP-producing neurons can be specifically stimulated by expressing hM3Dq and administering the ligand for hM3Dq, clozapine-N-oxide (CNO).

It can also be said that another embodiment of the present invention can be a kit for treating a neurological disease, comprising a recombinant vector containing a gene encoding a protein, preferably a membrane protein, more preferably a channelrhodopsin, for specifically stimulating thermoregulatory or metabolic regulatory neurons of a subject in response to light, preferably infrared light. With the present kit for treating a neurological disease, for example, a viral recombinant vector (e.g., adenovirus, adeno-associated virus, etc.) with a gene encoding channelrhodopsin operably linked to the QRFP promoter can be used to produce QRFP. It is possible to express channelrhodopsin in a neuron-specific manner and to stimulate QRFP-producing neurons specifically by irradiating them with light, preferably infrared light.

The recombinant vector included in the kit is not limited to any vector that can efficiently deliver genes to target cells (such as neurons). Such vectors include, for example, lentivirus vectors, adeno-associated virus vectors, adenovirus vectors, herpes virus vectors (e.g., herpes simplex virus vectors), poxvirus vectors, baculovirus vectors, papillomavirus vectors, viral vectors such as papova virus vector (e.g., SV40); plasmid vectors; phagemid vectors; cosmid vectors; bacteriophages such as lambda phage and M13 phage. Among these, adeno-associated virus vectors and adenovirus vectors are preferred, and adeno-associated virus vectors are more preferred.

The recombinant vectors can contain control sequences such as promoters, enhancers, ribosome-binding sequences, terminators, and polyadenylation sites so that the target gene can be expressed. In addition, if necessary, selection marker sequences such as drug resistance genes (e.g., kanamycin resistance gene, ampicillin resistance gene, puromycin resistance gene, etc.), thymidine kinase gene, diphtheria toxin gene, mCherry (red fluorescent protein), green fluorescent protein (GFP), (red fluorescent protein), green fluorescent protein (GFP), reporter gene sequences such as β-glucuronidase (GUS), FLAG, etc., can be included.

### EXAMPLES

Hereinafter, the present invention will be described in more detail with reference to examples, but the present invention is not limited by these examples.

### 1. Substantial Reduction of Neuronal Activities During OIH

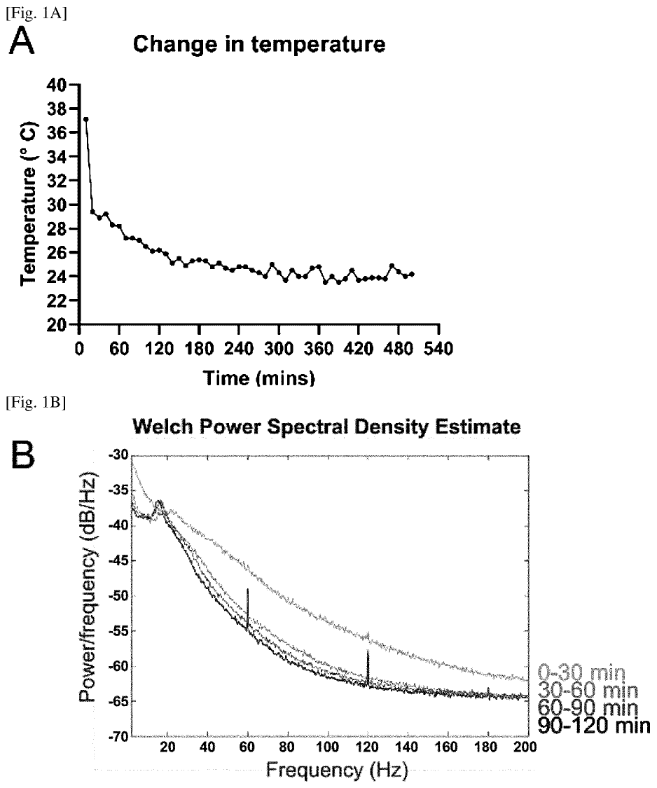

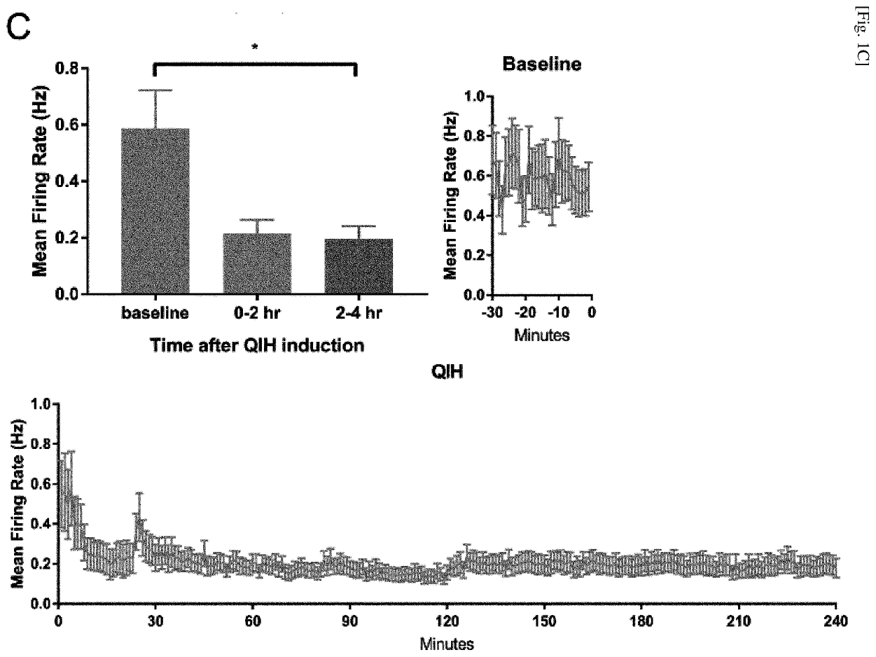

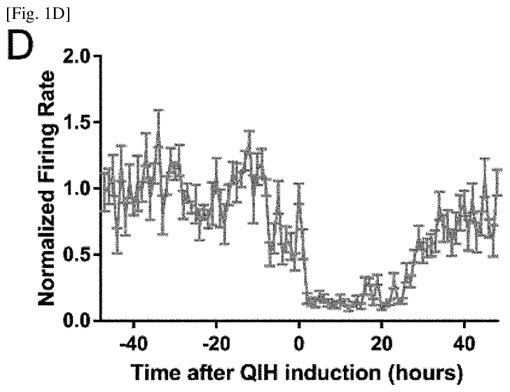

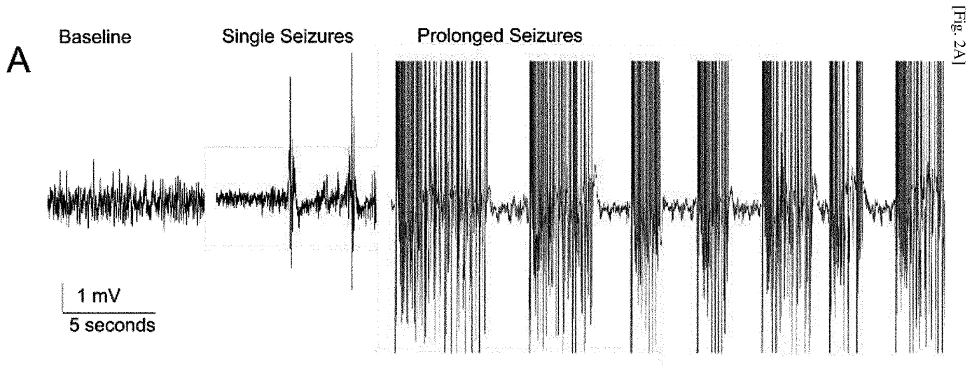

A previous study reported a low amplitude of electroencephalogram (EEG) signals acquired from the brain surface during QIH, suggesting an isoelectric state in the brain (Takahashi et al., 2020). To further examine the neuronal activity across different brain regions, we conducted extracellular tetrode recording in the hippocampus and the ventral part of the hypothalamus from mice under QIH. All the recording animals successfully entered QIH, as indicated by the reduction in their body temperature (FIG. 1A). Similar to the EEG result, power spectral density analysis of the local field potential (LFP) data acquired in the dorsal CA1 revealed a substantial reduction in broad frequency ranges (FIG. 1B). In line with the population activity, the individual pyramidal cells in the dorsal CA1 exhibited a rapid reduction in their spike activity after QIH induction (FIG. 1C). Similarly, the multi-unit activity recorded from the hypothalamus was also reduced upon induction of QIH (FIG. 1D). These results revealed a substantial reduction of neuronal activities in two different brain areas—the hippocampus and the hypothalamus—during the induced hypothermia and hypometabolism.

2. Delay in SRS Onset after OIH Induction During the Latent Period of TLE

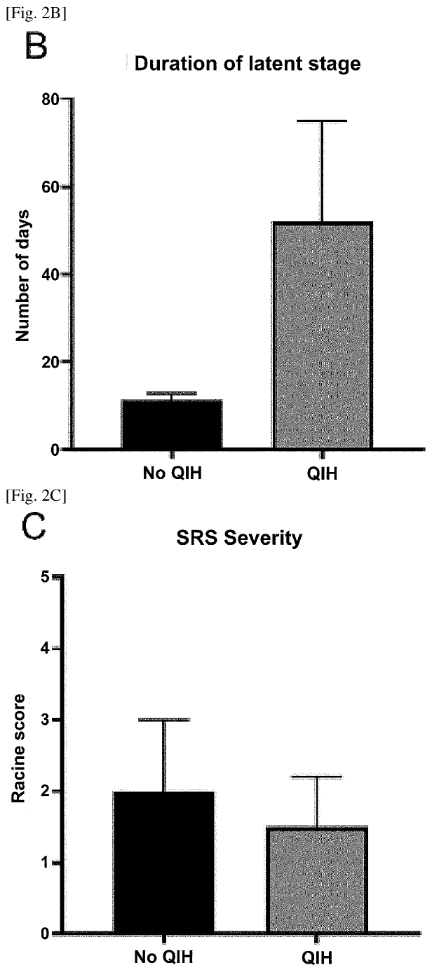

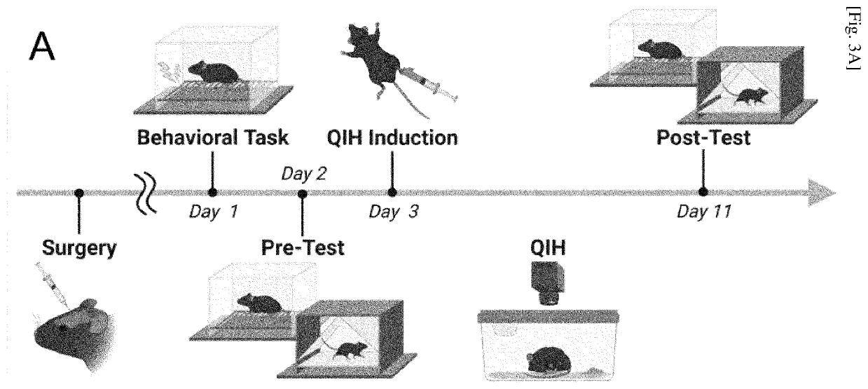

Taking advantage of the global suppression of neuronal activity during QIH, we tested the contribution of neuronal activity during the latent period to the later onset of SRS. Pilocarpine reliably induced seizures within thirty minutes after injection, with the prolonged seizure (status epilepticus) lasting for about two hours before termination with an anticonvulsant drug. LFP recording during this acute phase of epilepsy confirmed epileptic discharges and population spikes in the pyramidal cell layer of the dorsal CA1 (FIG. 2A). Twenty-four hours after midazolam injection to stop the acute seizure, QIH was induced by activating Q-neurons in the anteroventral periventricular nucleus, AVPe) in the hypothalamus, which is achieved by a interperitoneal injection of Clozapine-N-Oxide (CNO). After this injection, the mice stay for 48 hours under QIH, from which they spontaneously awake and rewarm to normal body temperatures. From then, animals were subject to daily monitoring in their homecages to detect the SRS onset.

QIH induction during the latent period produced a drastic delay in the SRS onset compared to control animals that stayed in the QIH chamber without induction (FIG. 2B). On the other hand, no difference in the severity in the SRS was observed between the two groups of mice (FIG. 2C). These findings suggest the long-lasting suppression effect of QIH on the development of epilepsy, representing a clear contrast with a previously invented cooling down approach and a strong therapeutic potential for controlling TLE.

3. QIH does not Influence Sensory Perception, Memory Recall, and its Behavioral Expression

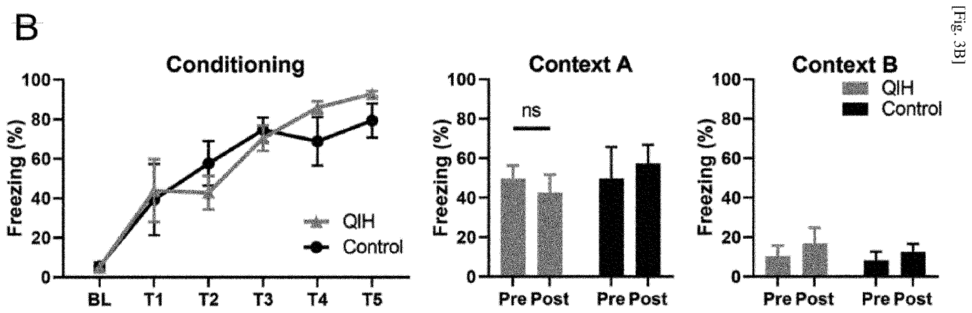

The state of hypothermia and hypometabolism involves various physiological and structural changes in the central nervous system, raising a possibility of side effects on the nervous control of vital functions, homeostasis, sensory processing, or cognitive functions. Despite these risks, hibernating animals maintain these CNS functions throughout their repeated cycles of hibernations, although their mechanisms are largely unknown. To determine a possible side effect of QIH, we trained mice using the contextual fear conditioning paradigm, which requires multimodal sensory processing, and tested the contextual fear memory after QIH (FIG. 3A). After training in the conditioning chamber (Context A), mice were first tested in the same context (Pre-Test A) and in a novel neutral context (Context B; Pre-Test B). The animals showed freezing behavior in Pre-Test A, but negligible degree of the fear response in Pre-Test B, indicating successful acquisition of the contextual memory (FIG. 3B). After 48 hours of QIH and 72 hours of the recovery period, their contextual memory was again tested in Context A and B (Post-Test A and Post-Test B). The group of animals that underwent QIH showed a comparable level of freezing behavior in Post-Test A to the control animals that stayed in the QIH chamber without induction (FIG. 3B). All groups of mice again showed a negligible degree of freezing, indicating a reliable discrimination of the shocked and neutral contexts. These results revealed intact sensory processing, memory recall, and its behavioral expression in the QIH animals during the testing sessions.

### Materials and Methods

### Animals:

Qrfp-iCre mice were provided by Dr. Takeshi Sakurai and Dr. Arisa Hirano group at the International Institute of Integrative Sleep Medicine (IIIS), Tsukuba University. All animals in our facilities were provided with water and food ad libitum, and maintained in a temperature- and humidity-controlled room with a 12 h light/dark cycle (lights on from 7:00 A.M. to 7:00 P.M.). All experimental protocols were approved by the Animal Care and Use Committee (ACUC) at the Okinawa Institute of Science and Technology Graduate University.

### Surgery:

All animals were single-housed prior to surgery. During surgery, mice were anesthetized with MMB (a cocktail of medetomidine, midazolam, and butorphanol) and 500 nL of AAV-hSyn-DIO-hM3D(Gq)-mCherry vector was infused through a small craniotomy above AVPe (ML ±0.3; AP +0.38; DV −5.0) at a rate of 50 nL per minute with a micropump syringe (World Precision Instrument). After infusion, the 35 G needle stayed at the infusion site for at least 15 minutes to allow diffusion of the vector. After surgery, animals were carefully monitored to full recovery. For LFP and single/multi-unit recording, we implanted a microdrive consisting of 9 independently adjustable tetrodes (14 um diameter, nichrome wire, gold plated to 200-300 kΩ). In the 2-3 weeks after surgery the depth of the tetrodes were slowly and manually adjusted until stable recording of LFP signals or separatable spike clusters was observed. All adjustments were conducted in a highly familiar small plastic bucket located in the recording room while the animals moved and behaved freely.

### QIH Induction:

Two to three weeks after surgery, mice were first habituated to intraperitoneal (i.p.) injection of saline four times (once per day). On the day of QIH induction, mice were allowed to be habituated to the experimental room (15C) for 1 hour. A dose of 1 mg/kg of clozapine N-oxide (CNO) was i.p. injected to induce QIH in the same experimental room under darkness and low ambient temperatures of about 18-19 degrees celsius. Mice were allowed to stay in the room for 48 hours, and then returned to the housing colony once the QIH state had wore off.

### Seizure Induction:

On the day of seizure injection, mice were first i.p. injected with 0.07 mL of scopolamine solution (0.5 mg/mL) to prevent adverse peripheral side effects. Thirty minutes later, 280 mg/kg pilocarpine solution was i.p. injected to produce acute seizure. After the mice reach stage 3, 4, or 5 on the Racine scale of behavioral seizures (modified version below), the development of status epilepticus (SE) is confirmed as a continuous state of seizures characterised by a behavioral expression such as a prominent head tic, rigidity of body and tail, and immobility. One to 3 hours after the initial onset of SE, 0.04 mL of Midazolam (5 mg/mL) was i.p. injected to attenuate the seizures.

TABLE 1
Modified Racine scale, slightly adapted
from Arshard and Naegele (2020)

| | Scale |
|---|---|
| Exhibited behavior | Score |
| Normal behavior | 0 |
| Immobilizations (freezing behavior) | 1 |
| Facial twitching and automatisms | 2 |
| Stiffened/extended tail, partial body clonus-forlimb or hindlimb | 3 |
| Rearing | 4 |
| Tonic-clonic with loss of posture | 5 |
| Death | 6 |

### Behavioral Monitoring of Seizure:

Animals are observed by trained personnel on a daily basis, starting with the first day after QIH exit. The monitoring process is performed while animals are in their homecages, in the experimental room. Observation is conducted for one hour per mouse per day using the scale above (table 1) to identify the severity of the behavioral seizures as they occur. For each mouse, the day on which the first seizure is observed, as well as the perceived severity, are recorded.

### Electrophysiology:

All data were acquired using 32-channel Digital Lynx 4SX acquisition system (Neuralynx). The local field potential (LFP) was filtered at 2-9000 Hz. Spike waveforms were filtered between 0.6-6 kHz and those above a peak threshold of 50 mV were time-stamped and digitized at 32,556 Hz. Spikes were manually sorted using SpikeSort3D software (Neuralynx). In short, waveform amplitudes of all spikes recorded from three channels were projected to three dimensional space and putative units were clustered. This procedure was repeated for all combinations of channels and also for energy of spikes. The raw local field potential (LFP) data were down-sampled using custom software written in C to 1627.8 Hz (a factor of 20), followed by quality control measures which excluded any signals that were either saturated or had a signal (4-12 Hz) to noise (45-55 Hz) ratio >6 dB. A low-pass filter with a cut-off frequency equal to half the target sampling frequency was applied to the LFP prior to downsampling to prevent signal distortion.

### Context Fear Conditioning:

Mice were first handled by an experimenter for at least 5 days to reduce their anxiety. During conditioning, the animals were first allowed to explore the context A (30.5L×24.1W×21H cm, metal grid floor, ethanol odor, white light, white noise) and then received 5 foot shocks (0.75 mA for 2 seconds with 30 seconds inter-shock-intervals). Next day, the same animals returned to the shocked context A and a novel neutral context B (30.5L×24.1W×21H cm, plastic flat floor, acetic acid odor, NIR light under darkness, triangle roof). After QIH induction and recovery, the mice were retested both in contexts A and B.

### Discussion

The current intervention is the first to describe the impact of artificially-induced hypothermia and hypometabolism on the development of TLE. For this sake, we used a recently-developed transgenic mouse line to produce QIH state and the pilocarpine model of TLE. In support of the hypothesis that the latent period after the acute seizure plays an active role for the onset of the later SRS, QIH induction during the latent period significantly delayed the transition to the chronic period of TLE. Given that Q-neurons in the hypothalamus are widely conserved across many mammalian species, including rodents, monkeys, and humans, it might be possible to generalize the application of QIH to the control of TLE over other mammalian species, including human patients. Furthermore, as Q-neurons are genetically defined as a subpopulation of neurons expressing pyroglutamylated RFamide peptide (QRFP) and identified using reverse pharmacology, it would be a fascinating attempt to achieve pharmacological manipulation of their activities without introducing transgenic interventions. Indeed, other approaches were demonstrated to be possible because we have recently succeeded in inducing QIH by optogenetic manipulation of Q-neurons in AVPe (Takahashi et al., 2022). These two lines of future studies would strengthen the benefits of the current invention.

One of the biggest problems with current medications for epilepsy is their short-term effects. Even if a patient identifies a medication that effectively suppresses seizures, it easily relapses once they stop taking the medication. As such, many people living with epilepsy typically have to take medication for life to manage their seizures. This problem is seen in contemporary manipulation of brain activity, such as optogenetics or the cooling methods described above. For example, Boldwin and Frost found a therapeutic effect of hypothermia (cooling down an animal's body with ice packs) on epilepsy, but they report spontaneous epileptic activity soon after warming the animal (Boldwin et al., 1956). Contrary to this widespread problem in prior attempts to cure epilepsy, our methods have a surprisingly long-term effect. In our experiments, we found the SRS several months after the induction of the hibernation-like state, as opposed to 1-2 weeks in the rodent model of the epileptic seizure. Furthermore, our study revealed suppression of neuronal activity across different brain areas during QIH (FIGS. 1C & D). This global effect is a strong advantage as a therapeutic approach for controlling the development of epilepsy since it does not require the identification of a seizure locus. It is expected that our invention has a strong potential for a novel therapeutic approach to epilepsy.

Although understanding how QIH suppresses the development of epilepsy is out of the scope of the current intervention, our preliminary data provide possible mechanisms for this effect. In hibernating animals, hibernation involves structural changes in the neuronal network and subsequent restoration and intact functions after arousal. Similarly, electron microscopy imaging revealed fewer synapses in the hippocampus during QIH (unpublished data). One can speculate this phenomenon is a drastic form of homeostatic plasticity that reverses the pathological state to its “default” mode. If hypothermia and hypometabolism are capable of resetting the network state in the brain, it might be possible to apply the same approach to other neurodegenerative and neurological disorders.

1. A method of treating a disease, the method comprising inducing hypothermia or hypometabolism in a subject in need thereof.

2. The method of treating a disease according to claim 1, the method comprising stimulating a thermoregulatory or metabolic regulatory neuron to induce hypothermia or hypometabolism in the subject.

3. The method of treating a disease according to claim 2, wherein the thermoregulatory or metabolic regulatory neuron is a Q neuron.

4. The method of treating a disease according to claim 1, wherein the disease is a neurological disease.

5. The method of treating a disease according to claim 4, wherein the neurological disease is epilepsy.

6. The method of treating a disease according to claim 5, wherein the epilepsy is temporal lobe epilepsy (TLE).

7. The method of treating a disease according to claim 5, wherein the inducing hypothermia or hypometabolism in the subject is performed during a latent phase of the disease.

8. The method of treating a disease according to claim 5, wherein the treating comprises delaying a seizure.

9. The method of treating a disease according to claim 5, wherein the treating comprises alleviating a seizure.

10. The method of treating a disease according to claim 1, wherein the subject is a mammal.

11. A kit for treating a neurological disease, comprising a recombinant vector comprising a gene encoding a protein that functions to specifically stimulate thermoregulatory or metabolic regulatory neurons and a compound for activating thermoregulatory or metabolic regulatory neurons.

12. The kit for treating a neurological disease according to claim 11, wherein the thermoregulatory or metabolic regulatory neurons are Q neurons.

13. The kit for treating a neurological disease according to claim 12, wherein the gene is a hM3Dq gene operably linked to a QRFP (pyroglutamylated RFamide peptide) promoter, and the compound is clozapine-N-oxide.

14-15. (canceled)

16. A method for treating a neurological disease, comprising administering to a subject in need thereof a recombinant vector comprising a gene encoding a photosensitive protein for specifically stimulating thermoregulatory or metabolic regulatory neurons in the subject in response to light, and irradiating said neurons with light.

17. The method of claim 16, wherein the photosensitive protein is channelrhodopsin, and wherein the gene encoding channelrhodopsin is operably linked to a QRFP promoter.

18. A method of treating a neurological disease, comprising administering to a subject in need thereof a compound for stimulating thermoregulatory or metabolic regulatory neurons in a subject and a recombinant vector comprising a gene encoding a receptor for specifically stimulating the thermoregulatory or metabolic regulatory neurons in response to the compound.

19. The method according to claim 18, wherein the recombinant vector comprises a hM3Dq gene and operably linked to a QRFP promoter, and the compound is clozapine-N-oxide.
